SEQUENCE LISTING (20 sequences)

SEQ ID NO: 1 - DNA, 32 bp, Homo sapiens
atctggcaccacaccttctacaatgagctgcg

SEQ ID NO: 2 - DNA, 32 bp, Homo sapiens
cgtcatactcctgcttgctgatccacatctgc

SEQ ID NO: 3 - DNA, 20 bp, Homo sapiens
cgaccatctgccgctttgag

SEQ ID NO: 4 - DNA, 20 bp, Homo sapiens
ccccctgtcccccattccta

SEQ ID NO: 5 - DNA, 18 bp, Homo sapiens
cccccggcggcaatagca

SEQ ID NO: 6 - DNA, 20 bp, Homo sapiens
tcggcgccggggagatacat

SEQ ID NO: 7 - DNA, 23 bp, Homo sapiens
ctacaacgcctacgagtcctaca

SEQ ID NO: 8 - DNA, 24 bp, Homo sapiens
gttgcaccagaaaagtcagagttg

SEQ ID NO: 9 - DNA, 20 bp, Homo sapiens
gcctcagcacctacctaccc

SEQ ID NO: 10 - DNA, 21 bp, Homo sapiens
ggttgcatgttcatggagtag

SEQ ID NO: 11 - DNA, 22 bp, Homo sapiens
tcaccccaacagagcgaagagg

SEQ ID NO: 12 - DNA, 24 bp, Homo sapiens
agagatttgatggaagggggtgtc

SEQ ID NO: 13 - DNA, 20 bp, Homo sapiens
cctccgctgggcttcattcc

SEQ ID NO: 14 - DNA, 23 bp, Homo sapiens
tgggggttctgcagtctttggtc

SEQ ID NO: 15 - DNA, 20 bp, Homo sapiens
gcggctactacaaccagagc

SEQ ID NO: 16 - DNA, 20 bp, Homo sapiens
gcacatggcacgtgtatctc

SEQ ID NO: 17 - DNA, 20 bp, Homo sapiens
ttggactatggctccgattc

SEQ ID NO: 18 - DNA, 20 bp, Homo sapiens
gatggcatcaggacaggact

SEQ ID NO: 19 - DNA, 20 bp, Homo sapiens
acagagctgcagtcccagat

SEQ ID NO: 20 - DNA, 20 bp, Homo sapiens
gtaggtggcgatctcgatgt

(19) United States
(12) Patent Application Publication
(10) Pub. No.: US 2008/0213387 A1
(43) Pub. Date: Sep. 4, 2008

(54) Cytotrophoblast Stem Cell

(75) Inventors: Harry MOORE, Sheffield (GB); Paul GERSKOWITCH, Sheffield (GB); Rosliah HARUN, Sheffield (GB)

Correspondence Address: MARSHALL, GERSTEIN & BORUN LLP, 233 S. WACKER DRIVE, SUITE 6300, SEARS TOWER, CHICAGO, IL, 60606, US

(73) Assignee: AXORDIA LIMITED, Sheffield (GB)

(21) Appl. No.: 11/873,234

(22) Filed: Oct. 16, 2007

Related U.S. Application Data
(63) Continuation of application No. PCT/GB2006/001273, filed on Apr. 6, 2006.

(30) Foreign Application Priority Data
Apr. 16, 2005 (GB) 0507755.7
Jun. 15, 2005 (GB) 0512170.2
Oct. 29, 2005 (GB) 0522101.5

(51) Int. Cl. A61K 35/12; C12N 5/08; C12N 5/22; C12N 5/02; A61K 35/34; A61P 41/00; A61K 35/54; C12N 15/06; A61K 35/32; A61K 35/23; A61K 35/39; C12Q 1/68; C40B 50/06; C12Q 1/02

(52) U.S. Cl. 424/548; 435/326; 435/366; 435/378; 435/6; 506/26; 435/29; 424/520; 424/556; 424/561; 424/572; 424/582; 424/549; 435/346; 435/449

(57) ABSTRACT

The invention relates to cytotrophoblast stem cells derived from embryonic stem cells; their differentiation into endovascular cytotrophoblast cells; and uses thereof.

The invention relates to cytotrophoblast stem cells derived from embryonic stem cells and uses thereof.

During mammalian development those cells that form part of the embryo up until the formation of the blastocyst are said to be totipotent (e.g. each cell has the developmental potential to form a complete embryo and all the cells required to support the growth and development of said embryo). During the formation of the blastocyst, the cells that comprise the inner cell mass are said to be pluripotential (e.g. each cell has the developmental potential to form a variety of tissues).

Embryonic stem cells (i.e. those having the characteristic of pluripotentiality) may be principally derived from two embryonic sources. Cells isolated from the inner cell mass are termed embryonic stem (ES) cells. In the laboratory mouse, similar cells can be derived from the culture of primordial germ cells isolated from the mesenteries or genital ridges of days 8.5-12.5 post coitum embryos. These would ultimately differentiate into germ cells and are referred to as embryonic germ cells (EG cells). Each of these types of pluripotential cell has a similar developmental potential with respect to differentiation into alternate cell types, but possible differences in behaviour (e.g. with respect to imprinting) have led these cells to be distinguished from one another, An indication that conditions may be determined which could allow the establishment of human ES/EG cells in culture is described in WO96/22362, which is incorporated by reference. The application describes cell lines and growth conditions which allow the continuous proliferation of primate ES cells which exhibit a range of characteristics or markers which are associated with stem cells having pluripotent characteristics.

During human implantation the continuous proliferation of cytotrophoblast stem cells (CTB) enables the embryo to rapidly invade the endometrial stroma and establish a haemochorial placenta. The early differentiation of cytotrophoblast to an invasive endovascular phenotype is critical for promoting feto-maternal immune tolerance and for remodelling uterine blood vessels and aberrant development is associated with serious complications of pregnancy, including recurrent miscarriage, pre-eclampsia (maternal high blood pressure) and restricted fetal growth (1-3). This process is poorly understood as investigations with human tissue are severely constrained by ethical and practical considerations.

In the mouse, trophoblast stem cells isolated from the pre- and post-implantation embryo can be maintained indefinitely in culture and have the capacity to differentiate along the trophoblast lineage (4). However, the derivation of human trophoblast stem cells from pre-implantation blastocysts has not been achieved, possibly due to the differences in early embryo development between these species (5).

We therefore used human embryonic stem cells HESCs) as a route to obtaining a trophoblast stem cell population. Wile HESCs differentiate spontaneously to trophoblast-like cells in cultures (5, 6), when supplemented with bone morphogenetic protein 4 (7) or when Oct 4 is down regulated (8), these cells are terminally differentiated and display a limited proliferative capacity. Trophoblast differentiation can develop further when HESCs are aggregated to form embryoid bodies (EBs) but residual HESCs and other cell types in the culture resulting from spontaneous differentiation confound the findings from these preparations.

We disclose the isolation of these isolated trophoblast-like cells and their maintenance in cell culture. We also disclose the use of these isolated trophoblast-like cells modulation of the immune system and in particular tissue engineering.

Tissue engineering or transplantation has implications with respect to many areas of clinical and cosmetic surgery. More particularly, tissue engineering relates to the replacement and/or restoration and/or repair of damaged and/or diseased tissues to return the tissue and/or organ to a functional state, For example, and not by way of limitation, tissue engineering is useful in the provision of skin grafts to repair wounds occurring as a consequence of: contusions, or bums, or failure of tissue to heal due to venous or diabetic ulcers. Furthermore, tissue engineering is also practised during: replacement of joints through degenerative diseases such as arthritis; replacement of coronary arteries due to damage as a consequence of various environmental causes (e.g. smoking, diet) and/or congenital heart disease including replacement of arterial/heart valve; repair of gastric ulcers; replacement bone tissue resulting from diseases such as osteoporosis; replacement muscle and nerves as a consequence of neuromuscular disease or damage through injury. In addition, organ transplantation has for many years been an established surgical technique to replace damaged and/or diseased organs. The replacement of heart, lung, kidney, liver, bone marrow, and double organ transplantation of, for example, heart and lung, are relatively common procedures.

However, in both tissue engineering and organ transplantation a major obstacle to the successful establishment of a tissue graft or organ transplantation is the host's rejection of the donated tissue or organ.

According to an aspect of the invention there is provided an isolated cytotrophoblast stem cell wherein said stem cell expresses HLA-G and HLA class I antigen.

In a preferred embodiment of the invention said stein cell is mononuclear.

In a further preferred embodiment of the invention said stem cell expresses at least one stem cell marker selected from the group consisting of: cytokeratin 7; stage specific embryonic antigen 1; human placental lactogen; caudal related homeobox; vimentin; and Cd9.

In a further preferred embodiment of the invention said stem cell is isolated from a primate, preferably a human.

In a preferred embodiment of the invention said stem cell is not a totipotent cell.

In a preferred embodiment of the invention said stem cell is genetically modified.

It will be apparent to the skilled person that cytotophoblast stem cells may be genetically modified by standard methods which enable the introduction of nucleic acid into a cell either by direct transfection of naked nucleic acid or vector nucleic acid. Alternatively human embryonic stem cells may first be modified and the genetically modified cytotrophoblast stems cells derived by methods hereindisclosed. A desirable genetically engineered trait would be to transfect human embryonic stem cells or cytotrophoblast stem cells with a nucleic acid encoding a marker gene, for example green fluorescent protein, to allow selection or identification.

According to a further aspect of the invention there is provided a composition comprising cytotrophoblast stem cells for use in tissue engineering.

According to a further aspect of the invention there is provided a culture comprising a cytotrophoblast stem cell according to the invention which culture is contained within a cell culture vessel.

According to a further aspect of the invention there is provided a spheroid body comprising a cytophoblast stem cell according to the invention and a collagen based cell support matrix.

In a preferred embodiment of the invention said cytotrophoblast stem cell in said spheroid body expresses at least one metalloprotease, preferably metalloprotease 2.

According to an aspect of the invention there is provided a method to derive human cytotrophoblast stem cells comprising selectively enriching for cytotrophoblast stem cells that express HLA-G and HLA class 1 antigen.

According to a further aspect of the invention there is provided a method to derive human cytotrophoblast stem cells from embryonic stem cells comprising the steps of:
i) forming embryoid bodies comprising cytotrophoblast cells in a cell culture vessel;
ii) identifying those embryoid body cultures which produce greater than about 500 m I.U./ml chorionic gonadotrophin;
iii) culturing the embryoid bodies identified in (ii) in conditioned media from fibroblast feeder cells;
iv) pooling those embryoid bodies which produce greater than about 500 m I.U./ml chorionic gonadotrophin and disaggregating said embryoid bodies; and
v) repeating (iv) until substantially all embryoid bodies produce high levels of chorionic gonadotrophin.

“Vessel” is defined as any means suitable to contain the above described cell culture. Typically, examples of such a vessel is a petri dish; cell culture bottle or flask; multiwell culture dishes.

In a preferred method of the invention said conditioned media comprises fibroblast growth factor 4 and heparin.

According to a further aspect of the invention there is provided spent medium produced by culturing the cytotrophoblast stem cells according to the invention.

According to a further aspect of the invention there is provided a method to produce endovascular cytotrophoblast cells comprising the steps of:
i) providing a preparation of cytotrophoblast stem cells;
ii) selecting from said preparation a population of cells that express both HLA-G and platelet endothelial cell adhesion molecule-1.

In a preferred method of the invention said selected cells further express Von Willebrand Factor.

According to a further aspect of the invention there is provided an endovascular cytotrophoblast cell obtained or obtainable by the method of the invention.

In a preferred method of the invention said preparation is cultured under high oxygen tension, preferably at least 5% CO₂.

According to a further aspect of the invention, there is provided an ill vitro method for the formation of spheroids comprising cytotrophoblast stem cells comprising:
i) providing a cell culture vessel comprising:
a) cytotrophoblast stem cells according to the invention;
b) cell culture medium; and
ii) providing conditions which promote the growth and differentiation of said cytotrophoblast stem cells in said spheroid.

According to a further aspect of the invention there is provided spent medium produced by culturing the spheroids comprising cytotrophoblast stem cells according to the invention.

According to a further aspect of the invention there is provided a method for the identification of genes associated with cytotrophoblast stem cell differentiation comprising the steps of:
i) providing a preparation comprising at least one cytotrophoblast stem cell according to the invention;
ii) extracting nucleic acid from said cell preparation;
iii) contacting said extracted nucleic acid with a nucleic acid array; and
iv) detecting a signal which indicates the binding of said nucleic acid to a binding partner on said nucleic acid array,

Preferably said method includes the additional steps of.
i) collating the signal(s) generated by the binding of said nucleic acid to said binding partner;
ii) converting the collated signal(s) into a data analysable form; and optionally
iii) providing an output for the analysed data.

In a further preferred method of the invention said method includes a comparison of the array signal produced between different populations of cytotrophoblast stem cells isolated from different subjects.

According to a further aspect of the invention there is provided a method for the preparation of a library comprising cytotrophoblast stem cell specific gene expression products comprising the steps:
i) providing a preparation comprising at least one cytotrophoblast stem cell according to the invention;
ii) extracting nucleic acid from said cell preparation;
iii) preparing a cDNA from ribonucleic acid contained in said extracted nucleic acid; and
iv) ligating cDNA formed in (iii) into a vector.

In a preferred method of the invention said vector is a phage based vector.

According to a further aspect of the invention there is provided an in vitro method to analyse the invasive properties of cytotrophoblast stem cells comprising the steps of:
i) providing a spheroid according to the invention and endometrial tissue; and
ii) monitoring the invasive phenotype of cytotophoblast cells with respect to said endometrial tissue.

According to a further aspect of the invention there is provided a method to identify agents which modulate the angiogenic activity of endothelial cells comprising the steps of:
i) providing a preparation of endovascular cytotrophoblast cells according to the invention and an agent to be tested;
ii) determining the effect or not of the agent on the proliferation and/or motility of said endovascular cytotrophoblast cells.

In a preferred method of the invention said agent is an antagonist (e.g., an anti-angiogenic agent).

In an alternative method of the invention said agent is an agonist (e.g. a pro-angiogenic agent).

Angiogenesis, the development of new blood vessels from an existing vascular bed, is a complex multistep process that involves the degradation of components of the extracellular matrix and then the migration, cell-division and differentiation of endothelial cells to form tubules and eventually new vessels. Angiogenesis is involved in pathological conditions such as tumour cell growth; non-cancerous conditions such as neovascular glaucoma; inflammation; diabetic nephropathy; retinopathy; rheumatoid arthritis; inflammatory bowel diseases (eg Crohn's disease, ulcerative colitis); and psoriasis. Current endothelial cell-lines used in the analysis of angiogenesis are so called “HuDMECS” which are commercially available endothelial cells, The present invention provides a new model endothelial cell-line useful in the study of angiogenesis.

According to an aspect of the invention there is provided the use of a cytotrophoblast cell, or a cell derived from a cytotrophoblast cell, in the manufacture of a cell composition for use in the modulation of the immune system.

According to a further aspect of the invention there is provided the use of a cytotrophoblast cell or a cell derived from a cytotrophoblast cell, in the manufacture of a cell composition for use in the modulation of cell/tissue rejection in transplantation therapy.

In a preferred embodiment of the invention said cell is a mammalian cell, preferably a human cell.

According to a further aspect of the invention there is provided a composition comprising an isolated mammalian cytotrophoblast cell, or a cell derived from a cytotrophoblast cell, and at least one further isolated mammalian cell that is not a mammalian cytotrophoblast cell.

In a preferred embodiment of the invention said mammalian cell is a human cell.

In a further preferred embodiment of the invention said cell is selected from the group consisting of: an epidermal keratinocyte; a fibroblast (e.g. dermal, corneal; intestinal mucosa, oral mucosa, bladder, urethral, prostate, liver) an epithelial cell (e.g. corneal, dermal, corneal; intestinal mucosa, oral mucosa, bladder, urethral, prostate, liver); a neuronal glial cell or neural cell; a hepatocyte stellate cell; a mesenchymal cell; a muscle cell (cardiomyocyte, or myotube cell); a kidney cell; a blood cell (e.g. CD4+ lymphocyte, CD8+ lymphocyte; a pancreatic β cell; or an endothelial cell.

In a preferred embodiment of the invention said cell is a pancreatic β cell.

In a further preferred embodiment of the invention said mammalian cell is a stem cell.

In a preferred embodiment of the invention said stem cell is selected from the group consisting of: a haemopoietic stem cell; a neural stem cell; a bone stem cell; a muscle stem cell; a mesenchymal stem cell; an epithelial stem cell (derived from organs such as the skin, gastrointestinal mucosa, kidney, bladder, mammary glands, uterus, prostate and endocrine glands such as the pituitary): an endodermal stem cell (derived from organs such as the liver, pancreas, lung and blood vessels); an embryoic stem cell; an embryonic germ cell.

In a preferred embodiment of the invention said mammalian cell is an embryonic stem cell or an embryonic germ cell.

In a further preferred embodiment of the invention said mammalian cell and said cytotrophoblast cell are autologous.

Preferably said embryonic stem cell/embryonic germ cell are autologous with said cytotrophoblast cell.

In a farther preferred embodiment of the invention said composition comprises an additional agent wherein said agent is an immunosuppressant.

According to a farther aspect of the invention there is provided a vehicle wherein said vehicle includes a mammalian cytotrophoblast cell, or a cell derived from a cytotrophoblast cell, and at least one further isolated mammalian cell that is not a mammalian cytotrophoblast cell.

Vehicle is defined as any structure to which cells may attach and proliferate. Examples include a prosthesis, implant, matrix, stent, gauze, bandage, plaster, biodegradable matrix and polymeric film. Matrix material may be synthetic or naturally occurring and either long-lasting or biodegradable.

According to a further aspect of the invention there is provided a method to modulate cell/tissue rejection in transplantation therapy comprising:
i) surgically inserting into an animal a cytotrophoblast cell, or a cell derived from a cytotrophoblast cell and at least one further mammalian cell; and optionally
ii) monitoring the immune status of the animal as a measure of the acceptance or otherwise of said mammalian cell.

In a preferred method of the invention said mammalian cell is a human cell.

In a farther preferred method of the invention said cell is selected from the group consisting of: an epidermal keratinocyte; a fibroblast (e.g. dermal, corneal; intestinal mucosa, oral mucosa, bladder, urethral, prostate, liver) an epithelial cell (e.g. corneal, dermal, corneal; intestinal mucosa, oral mucosa, bladder, urethral, prostate, liver); a neuronal glial cell or neural cell; a hepatocyte stellate cell; a mesenchymal cell; a muscle cell (cardiomyocyte, or myotube cell); a kidney cell; a blood cell (e.g. CD4+ lymphocyte, CD8+ lymphocyte; a pancreatic β cell; or an endothelial cell.

In a further preferred method of the invention said cell is a pancreatic β cell.

In a preferred method of the invention said mammalian cell is a stem cell.

In a preferred method of the invention said stem cell is selected from the group consisting of: a haemopoietic stem cell; a neural stem cell; a bone stem cell; a muscle stem cell; a mesenchymal stem cell; an epithelial stem cell (derived from organs such as the skin, gastrointestinal mucosa, kidney, bladder, mammary glands, uterus, prostate and endocrine -lands such as the pituitary); an endodermal stem cell (derived from organs such as the liver, pancreas, lung and blood vessels); an embryonic stem cell; an embryonic germ cell.

In a preferred method of the invention said mammalian cell is an embryonic stem cell or an embryonic germ cell.

In a further preferred method of the invention said mammalian cell and said cytotrophoblast cell are autologous.

According to a further aspect of the invention there is provided an isolated chimeric cell wherein said cell is the product of a fusion between a first cell, or part thereof which is a cytotrophoblast cell that expresses HLA-G and HLA class I antigen and a second cell wherein said first and second cell are derived from the same species. Methods that promote the fusion of cells are well known in the art (Kennett, R. H. (1979). Cell Fusion in: Cell Culture, Methods in Enzymology. (eds. Jakoby, W. B., and Pastan, I. H.) Academic Press San Diego, 58, 345-359 which is incorporated by reference in its entirety). It is also well known that cell hybrids may be formed by fusing the cytoplasm of a cell (in which the nucleus has been removed) with a selected intact cell to form a so called cybrid (Ege, T., Zeuthen, J., Ringertz, N. R. (1973). Cell fusion with enucleated cytoplasms. Nobel, 23, 189-194; Veomett, G., Prescott, D. M., Shay, J., Porter, K. R. (1974). Reconstruction of mammalian cells from nuclear and cytoplasmic components separated by treatment with cytocholasin B. Proc Nat Acad Sci, 71, 1999-2002; Wright, W. E., and Hayflick L. (1975). Use of biochemical lesions for selection of human cells with hybrid cytoplasms. Proc. Nat. Acad. Sci (USA). 72, 1812-1816 which are incorporated by reference in their entirety). This has enabled investigation into nucleo-cytoplasmic interactions and, in particular, the influence of cytoplasmic determinants on nuclear gene expression. It has been known for several years that selected chemical treatments of cells in culture can result in cells extruding nuclei resulting in the formation of separate nuclear and cytoplasmic parts termed karyoplasts and cytoplasts, respectfully. These sub-cellular components have been used in fusion experiments. For example, it is possible to produce a cytoplast from one cell and fuse the cytoplast to a selected cell to form a cytoplasmic hybrid or cybrid, In addition it is also possible to fuse the karyoplast or cell with a selected cell to form a nuclear hybrid. The nuclei fuse after nuclear membrane breakdown during mitosis and reconstitute after cytolinesis to form a polyploid or anueploid nucleus. The fusion of embryonal stem cells is described in Duran C, Talley P J, Walsh J, Pigott C, Morton I E, and Andrews P W. Hybrids of pluripotent and nullipotent human embryonal carcinoma cells: partial retention of a pluripotent phenotype. Int J Cancer. Aug. 1, 2001 ; 93(3):324-32 which is incorporated by reference in its entirety.

In a preferred embodiment of the invention said chimeric cell comprises a cytoplasmic part derived from a cytotrophoblast cell and a nucleus derived from a cell that is not a cytotrophoblast cell.

In an alternative preferred embodiment of the invention said chimeric cell comprises a nucleus derived from a cytotrophoblast cell and a cytoplasmic part derived from a cell that is not a cytotrophoblast cell.

In a preferred embodiment of the invention the first and second cells are human cells.

In a further preferred embodiment of the invention said second cell is selected from the group consisting of: an epidermal keratinocyte; a fibroblast (e.g. dermal, corneal; intestinal mucosa, oral mucosa, bladder, urethral, prostate, liver) an epithelial cell (e.g. corneal, dermal, corneal; intestinal mucosa, oral mucosa, bladder, urethral, prostate, liver); a neuronal glial cell or neural cell; a hepatocyte stellate cell; a mesenchymal cell; a muscle cell (cardiomyocyte, or myotube cell); a kidney cell; a blood cell (e.g. CD4+ lymphocyte, CD8+ lymphocyte; a pancreatic β cell; or an endothelial cell.

According to a further aspect of the invention there is provided a cell culture comprising a chimeric cell according to the invention.

According to a further aspect of the invention there is provided a method for the production of a chimeric cell comprising the steps of:
i) forming a preparation comprising a first cell which is a cytotrophoblast cell that expresses HLA-G and HL-A class I antigen and a second cell wherein said first and second cells are derived from the same species; and
ii) providing conditions wherein said first and second cells fuse to form a chimeric cell.

According to a further aspect of the invention there is provided a chimeric cell according to the invention for use in the manufacture of a cell composition for the modulation of cell/tissue rejection in transplantation therapy.

According to a further aspect of the invention there is provided a method to treat a condition that would benefit from transplantation therapy comprising administering a chimeric cell according to the invention.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following Figures:

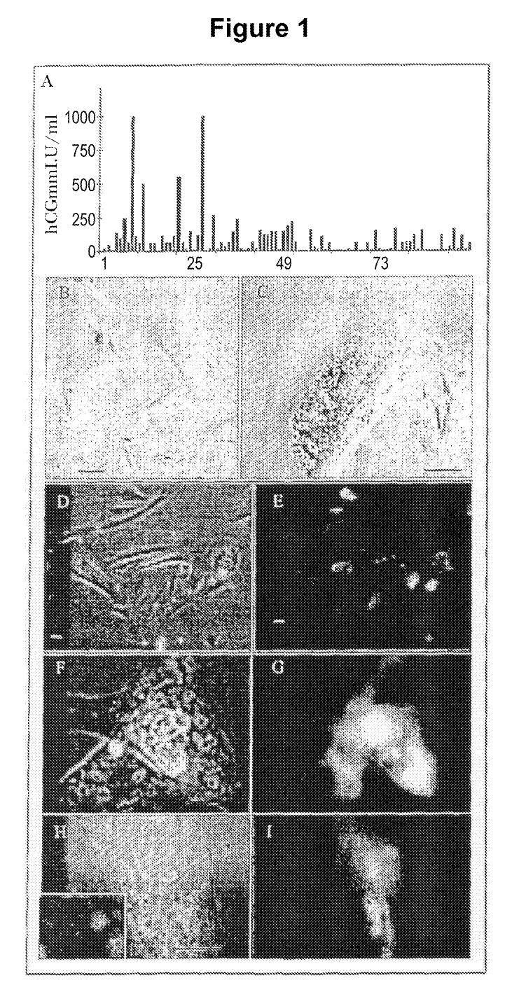

FIG. 1 illustrates the derivation and initial characteracterisation of human CTBS cell lines; (A) Histogram of hCGβ concentration in culture medium in 96-wells containing sin-le embryoid bodies.(B)Adherent epithelial CTBS cells in ‘TS’ culture without feeder cells. Bars=20 μm throughout. (C) Adherent multinucleated syncytiotrophoblast in same culture as (B). (D) & (E) adherent CTBS cells under phase contrast and UV light displaying cytokeratin (green) and hCGβ (red/orange) immunolocalisation (nucleus, blue). (F) & (G) Single CTBS cells fusing to adherent multinucleated syncytium under phase contrast and UW. Single cells mainly cytokeratin⁺ and syncytium mainly :hCGβ⁺. (H) Adherent GFP-syncytial trophoblast (phase contrast and UV) and (I) UV alone. Inset low power of GFP-trophoblast vesicles;

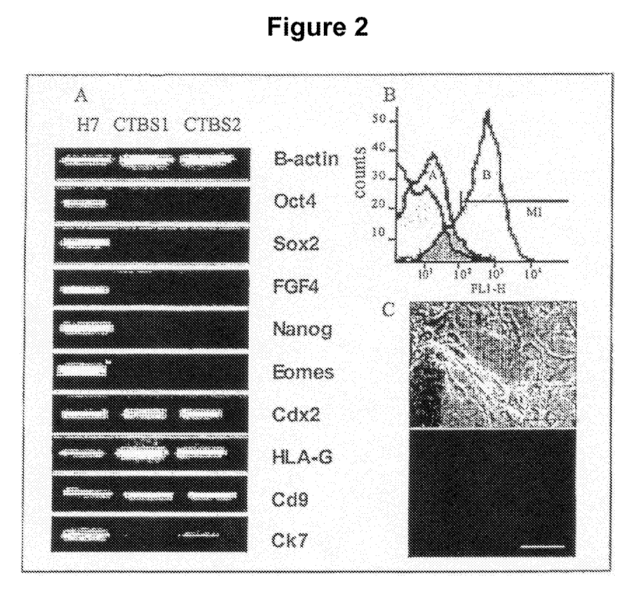

FIG. 2 illustrates RT-PCR and FACS analysis of TS cells. (A) Gene expression (RT-PCR) for undifferentiated HESCs (H7) and CTBS 1 and 2 cell lines. (B)FACS analysis profile for CTBS2 (similar data for CTBS1 not shown) in early culture. A proportion of cells express non classical HLA-G (peak A) while the majority express all forms of HLA class 1. Phase-contrast and immunofluorescent labelling of cells used for FACs analysis indicating HLA-G staining. Bar=20 μm;

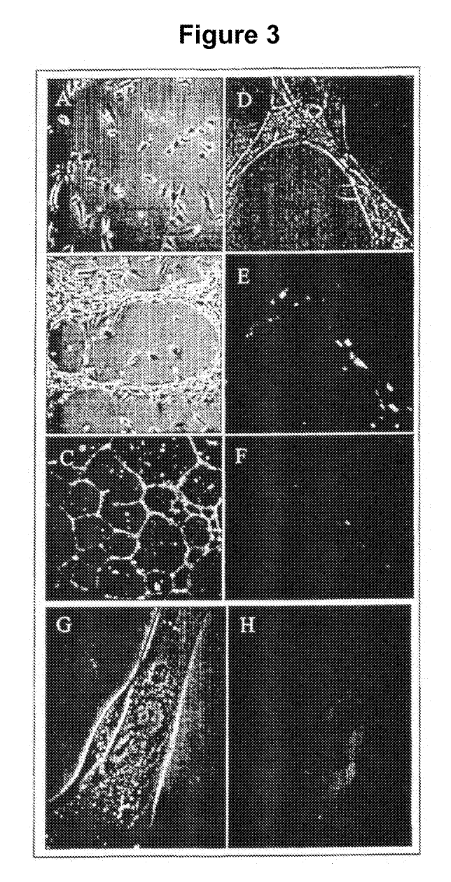

FIG. 3 illustrates the differentiation of CTBS cell line to endovascular cells in ‘TS’ conditioned medium. A) Phase contrast micrograph of single adherent cytotrophoblast of CTBS1 cell line. B) The cells in (A) after 1-2 weeks in culture. Long aggregates display typical endothelial-like morphology. (C) Dark field, low power micrograph of culture flask. D) Phase-contrast of endovascular cell aggregate displaying co-expression of HLA-G (B) and PECAM-1 (F). (G) Phase contrast of multinucleated ‘giant’ adjacent to endovascular cell. E) E.cadherin immunolocalisation was much greater in giant cells than endovascular cells; and

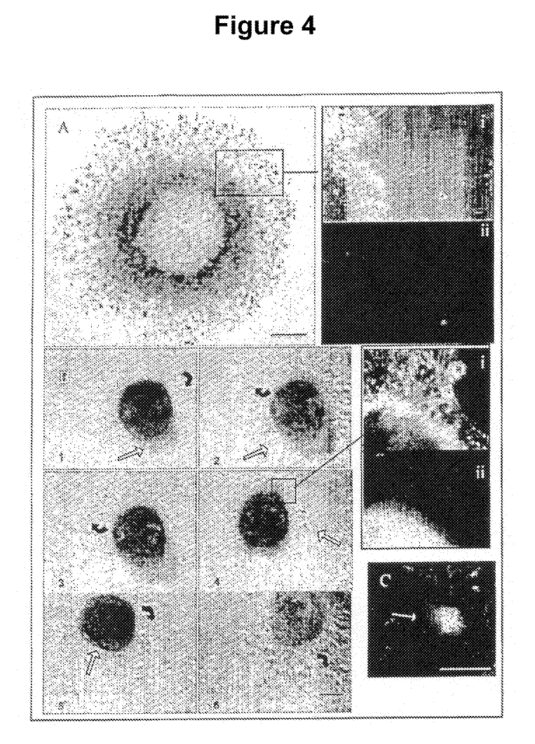

FIG. 4 illustrates CTBS spheroids in extracellular matrix and endometrial co-culture. (A) CTBS spheroid (CTBS1 cell line) in Matrigel culture for 5 days with long microvilli protrusions of syncytium. Inset (i) and (ii): higher magnification phase contrast and immunostaining displaying cytokeratin(green) and hCGβ (red) in syncytial bed. Bar=100 μm. (B) Images from time-lapse movie (see supplementary information) of CTBS1 aggregate in co-culture with endometrial stromal cells; bar=150 μm. Black arrow throughout indicates direction of migration of vesicle. (1), white arrow indicates invasive cytotrophoblast outgrowth; (4 and 5), white arrow indicates stromal erosion site; (4) inset (i) and (ii), higher magnification of margin at erosion site showing phase contrast and MMP-2 immunolocalisation. (C) CTBS-GFP cells in co-culture with endometrial stroma; bar=20 μm.

### MATERIALS AND METHODS

### Reverse Transcription and Polymerase Chain Reaction (RT-PCR).

Polymerase chain reaction (PCR) technique is used to identify genetic markers that are characteristic to cell type. Total RNA (2 μg) was reverse transcribed using 1 μg oligo-dT primer with MMLV Reverse-Transcriptase (Promega) in a 40 μl reaction volume containing 1.25 mM dNTPs at 37° C. PCR was performed using 1 μl of CDNA in 50 μl reaction volume containing 15 pmol of each primer, 0.2 mM dNTPs and 1 unit Taq polymerase (Promega). Primer sequences used and conditions of these reactions were as follows:

β-actin-F: 5′-ATCTGGCACCACACCTTCTACAATGAGCTGCG-3′;
β-actin-R: 5′-CGTCATACTCCTGCTTGCTGATCCACATCTGC-3′
(60° C. annealing, x23 cycles).
Oct4-F: 5′-CGACCATCTGCCGCTTTGAG-3′;
Oct4-R: 5′-CCCCCTGTCCCCCATTCCTA-3′
(60° C. annealing, x23 cycles).
Sox2-F: 5′-CCCCCGGCGGCAATAGCA-3′;
Sox2-R: 5′-TCGGCGCCGGGGAGATACAT-3′
(60° C. annealing, x38 cycles).
FGF4-F: 5′-CTACAACGCCTACGAGTCCTACA-3′
FGF4-R: 5′-GTTGCACCAGAAAAGTCAGAGTTG-3′
(56° C. annealing, x40 cycles).
Nanog-F: 5′-GCCTCAGCACCTACCTACCC-3′
Nanog-R: 5′-GGTTGCATGTTCATGGAGTAG-3′
(60 annealing and x30 cycles).
Eomes-F: 5′-TCACCCCAACAGAGCGAAGAGG-3′;
Eomes-R: 5′-AGAGATTTGATGGAAGGGGGTGTC-3′
(57° C. annealing, x35 cycles).
Cdx2-F: 5′-CCTCCGCTGGGCTTCATTCC-3′;
Cdx2-R: 5′-TGGGGGTTCTGCAGTCTTTGGTC-3′
(60° C. annealing, x35 cycles);
HLA-G-F: 5′-GCGGCTACTACAACCAGAGC-3′;
HLA-G-R: 5′-GCACATGGCACGTGTATCTC-3′
(55° C. annealing, x26 cycles).
CD9-F: 5′-TTGGACTATGGCTCCGATTC-3′;
CD9-R: 5′-GATGGCATCAGGACAGGACT-3′
(55° C. annealing, x26 cycles).
CK7-F: 5′-ACAGACCTGCAGTCCCAGAT-3′;
CK7-R: 5′-GTAGGTGGCGATCTCGATGT-3′
(55° C. annealing, x26 cycles).

Fluorescence activated cell sorting (FACS) Troplioblast cells were prepared for cell sorting by dissociating CTBS cells into single cells with Trypsin-EDTA, Cells were resuspended at 5×10⁶/ml in FACS buffer with 40% normal goat serum to block on ice for 20 minutes. 90 μl of the cell suspension were aliquoted into FACS tube and 10 μl of G233 (TS marker for HLA-G) and W6/32 HLA-Class I control was added. G233 supernatant with NaN₃ (mouse IgG₂ₐ) was kindly given by Dr Ashley King, University of Cambridge. The cells were incubated on ice for 30 minutes. After incubation, the cells were washed twice before being labelled with anti-mouse polyvalent immunoglobulin FITC conjugate for 30 minutes on ice. The cells were washed again and resuspended in 300 μl buffers
Determination of hCGβ Concentration in Cell Cultures.

Concentrations of hCGβ were determined using a sandwich enzyme immunoassay kit (Cat. # EIA-1469, DRG Diagnostics). The standards and the samples were incubated with 100 μl anti-hCG enzyme-conjugate for 30 minutes at room temperature followed by a five times washing procedure. A second incubation with 100 μl substrate solution for 10 minutes was stopped with the addition of 50 μl stop solution. Absorbance was read at 450±10 nm with a microtitre plate reader. The concentration of hCGβ in the samples was determined from the standard curve as m I.U./ml.

Constitutive Expression of eGFP in HESCs

A pCAG-GFP expression vector was constructed by excision of eGFP from pEGFP-1 (Clontech) with XhoI and NotI and insertion into the pCAG vector¹⁶. H7 cells were seeded at the equivalent of 2×10⁵ per well of a 6-well plate on Matrigel. The next day they were transfected using ExGen 500 (Fermentas) according to the manufacturer's instructions. The DNA/NaCl Exgen mixture was then added directly to the normal growth medium in the well. The plate was centrifuged at 280 g for 5 minutes and placed back in the incubator. The medium was exchanged the next day with hES growth medium containing puromycin (at 1 μg/ml). Viable colonies were picked after 2-3 weeks.

### Endometrial—CTBS Spheroid Co-Culture.

Luteal phase endometrial biopsies were obtained from women undergoing hysterectomy under full ethical approval and patient consent, Endometrial epithelial and stromal cells were isolated using a method described previously²⁴. Preparations were embedded in Matrigel on membrane inserts and primed with progesterone for 24 hours before the start of co-culture with CTBS. In monolayer co-culture, CTBS spheroids were cultured on a confluent layer of stromal or epithelial cells in culture wells. The co-cultures were maintained in 500 μl of either conditioned TS medium or serum-free HES medium up to six days.

### Time-Lapse Microscopy

Adherent CTBS cultures or CTBS-endometrial co-cultures were continuously monitored under an inverted microscope in a humidified physiological chamber at 37° C. in 5% CO₂ in air (DigitalPixel Ltd) for up to three days, Preliminary experiments indicated no difference in the viability of cells maintained under these conditions compared to a standard incubator. Regions of interest (ROI) were identified and programmed for analysis using Simple PCI software (C-Imaging) with control over xyz scan, transmitted light, and image capture. Routinely 20 ROIs were identified with image capture every 15 minutes.
1. Movie of adherent multinuclear TS cells exhibiting cell fusion
2. Movie of TS vesicle attached and migrating on endometrial stromal cells in co-culture and displaying erosion site.

### EXAMPLE 1

First, we generated trophoblast-containing EBs, using HESCs (H7 and H14) of normal karyotype, which were maintained and passaged by standard protocols using serum-replacement medium (8,9). EBs were prepared by aggregation of single HESCs (dissociated with 1 mg/ml collagenase IV) in ES medium without basic fibroblast growth factor in Petri dishes in 5% CO₂ in air. On day 5, EBs were then transferred singly to wells of a 96-well culture plate and cultured for a further three days before aliquots of medium were subjected to ELISA assay as described previously (10). HCGβ was detected in most wells (4×96 well plates) but only 3.8% of wells had concentration of hormone greater than 500 m I.U./ml (FIG. 1A). The EBs in these wells were of equivalent size and morphology, indicating that any increase in hCGβ was likely to be due mainly to the proportion of trophoblast cells rather than a greater overall number of cells.

### EXAMPLE 2

To select for CTBS cells, EBs exhibiting high hCGβ secretion were subjected to several rounds of selective enrichment by growth in ‘TS’ medium comprising conditioned medium from fibroblast feeders supplemented with fibroblast growth factor 4 (FGF4) and heparin. TS medium promotes differentiation of murine trophoblast stem cells from extra-embryonic ectoderm (4). Those EBs maintaining a high secretion of hCGβ were pooled, disaggregated and allowed to form new EBs and this enrichment protocol repeated consecutively for three rounds until all EBs displayed consistently high hCGβ secretion. EBs were disaggregated (0.25% trypsin-EDTA) and then single cells allowed to proliferate in adherent culture in TS medium without feeders. Control cultures of EBs in HES medium without bFGF exhibited only basal hCGβ levels indicating poor trophoblast differentiation. Initially, four cell lines were generated with variable proliferation, two of which have maintained persistent proliferative capacity for more than 30 passages (CTBS1 from H7 HESC and CTBS2 from H14 HESC) and form epithelial-like cell colonies with single and multinucleated cells (FIG. 1B). An additional CTBS cell line (CTBS-GFP1) was generated by the same methods but from H7 HESCs with constitutive expression of enhanced green fluorescent protein, eGFP (11) (FIGS. 1H and 1I). Continuous proliferation of each cell line was related to the persistence of a mononuclear cytotrophoblast population (relative to syncytium formation) as determined by immunostaining for cytokeratin and hCGβ (FIG. 1D-G). Cell proliferation was maintained by regular cell passage every 5 days as this inhibited terminal differentiation. When CTBS cells were aggregated and returned to mouse embryonic fibroblast feeders with HES medium they failed to revert to or generate either HESC colonies or EBs with pluripotent developmental capacity other than trophoblast. This indicated the absence of residual HESCs in the cell lines and the likely restricted developmental capacity of CTBS cells as has been shown for the mouse (10).

### EXAMPLE 3

We confirmed the trophoblast phenotype of the cell lines by immunolocalisation of the pan trophoblast marker cytokeratin 7 (12, 13), Stage-Specific Embryonic Antigen 1 (SSEA1, 4), and human placental lactogen (hPL, 14). Additionally, reverse transcription and the polymerase chain reaction (RT-PCR) were performed with primer sequences for marker genes of HESCs and trophoblast. Compared with HESCs, mRNA expression for Oct 4, Sox2, FGF4, Nanog in the derived cell lines was absent while trophoblast-related mRNAs for Cdx2 (caudal-related homeobox), Ck7, HLA-G, and Cd9 and were up regulated or maintained (FIG. 2A). The latter two are known markers for extravillous cytotrophoblast which invades the uterine stroma (deciduas) during placentation (15). Surprisingly, eomesodermin (eomes), a marker of mouse early postimplantation trophoblast, was expressed strongly in HESCs but was weak or absent in the CTBS cell lines (FIG. 2A). Several reports have highlighted differences between mouse and human ESCs (4,16) including eomesodermin expression in HESCs but not mouse ES cells (16). Furthermore, the expression of some trophoblast markers in stock cultures of HESCs may relate to spontaneous differentiation to trophoblast lineage. We had previously shown that expression of trophectodermial markers in such cultures occurred predominantly in the SSEA (−) and SSEA1 (+) subsets of cells, consistent with their expression in the differentiated derivatives of the HESCs rather than in the HESCs themselves (4,16).

### EXAMPLE 4

To further assess the subtype of trophoblast cells, the comparative cell surface expression of histocompatibility HLA class I (an HLA antibody W6/32) and HLA-G (antibody G233) antigens was determined by fluorescent activated cell sorting (FACS) and immunolocalisation (14, 15, and 17). The majority of cells (˜90%) expressed HLA-class I histocompatibility antigens consistent with extravillous trophoblast (4, 15) (FIG. 2B). The expression of HLA-G (18), the non-classical HLA-class I antigen also specifically expressed in anchoring extravillous cytotrophoblast of first trimester placentae (14, 15) was relatively weak in most cells, but a small proportion (10%) of cells displayed strong immunoreactivity (FIGS. 2B and C). Some cells expressed vimentin, possibly indicating interstitial CTB (14). Following extended culture for one week or more in T25 flasks, the proportion of HLA-G⁺ cells increased considerably (>90%). These cells exhibited distinct endothelial cell morphology similar to cultures of differentiating cytotrophoblast from first trimester human placental tissue (15) and resembled endotlielial morphological differentiation from primate embryonic stem cells (19,20). Significantly, however, the cells co-expressed HLA-G and the platelet endothelial cell adhesion molecule-1 (PECAM-1), both markers of invasive endovascular (endothelial-like) CTB (3,21; FIG. 3) E-cadherin immunolocalisation was weak on endovascular cells but strong on a relatively small proportion (<5%) of multinucleated cells also present at this stage and most likely equivalent to the syncytial giant cells found in stroma of the developing placenta.

### EXAMPLE 5

To determine the functional capacity of CTB cells, we first investigated the formation of non-proliferative, syncytiotrophoblast by cell-cell fusion of villous cytotrophoblast (1). The spontaneous generation of syncytium in adherent cell cultures of CTBS1 was monitoring cells under an inverted microscope for up to 3 days in a chamber at 37° C. in 5% CO₂ in air by continuous time-lapse recording. Adherent cells displayed progressive migration across the culture dish promoted by pseudopodial-like extension of cells. When cells occasionally converged they fused to form multi-cellular syncytiotrophoblast cells (FIG. 1G) that were hCGβ, and Ck7 positive but HLA class 1 negative. This cell fusion was captured unequivocally by digital time-lapse microscopy.

### EXAMPLE 6

Next, we examined the invasive implantation potential of the CTBS cell lines by adopting a three-dimensional spheroid culture. This technique has been shown to maintain extra villous CTB phenotype of first trimester placental tissue (22). Aggregates of CTBS cells were generated from confluent monolayers following brief trypsinisation and incubation in non-adherent culture for 5-10 days. When cultured in extracellular matrix (Matrigel) drops, these CTBS spheroid aggregates developed characteristic outgrowths, which expressed hCGβ and cytokeratin (FIG. 4Ai and ii) The hCG receptor is expressed on invasive cytotrophoblast and similar observations have been reported for EB differentiation to trophoblast (15). On further culture with primary human endometrial tissue (luteal phase) prepared using well-characterised protocols (23), CTBS aggregates attached to both epithelial cells and stromal cells. Significantly, as shown by time-lapse microscopy (FIG. 4B) CTBS spheroids with stromal cell cultures displayed a characteristic circular migratory movement and exhibited polar outgrowths from which endovascular cells streamed. After about 24-36 hours in co-culture, these trophoblast outgrowths were the site of an erosion of the extracellular matrix of the stroma (and supplementary information, movie 2). This was identified by the rapid retraction of the trophoblast vesicle due to the dissolution of underlying extracellular matrix of the stromal cells (FIG. 4B, 2-5). A similar process of trophoblast invasion has been observed for human blastocyst co-culture with stromal cells in vitro (24). The erosion site was characterised by extravillous (HLA-G⁺) trophoblast that expressed matrix metalloproteinase 2 (gelatinase A, FIG. 4b4, i and ii), identified recently as a key enzyme correlated with first trimester invasive capacity of human cytotrophoblast (25) and whose activity is altered in cytotrophoblast in women with pre-eclampsia (26). Single GFP- trophoblast cells with endometrial stroma in culture displayed a similar response.

These CTBS cell lines are the first distinct set of multipotent progenitor stem cell lines to be derived from HESCs and maintained independently. The method of selecting individual viable EBs with an appropriate secretory marker, followed by rounds of enrichment by the regeneration of EBs, could be applied in principle to derive a range of other cell types. It has been shown previously that clonally derived HESCs maintain full pluripotency and proliferation (27) suggesting that CTBS cells develop from a homogeneous HESC population rather than multiple (i.e. ES and TS) precursors. Hence, our findings differ from the mouse where trophoblast cells may be derived from extra-embryonic ectoderm but not from murine ESCs without conditional gene expression (28).

We have derived, for the first time, human cytotrophoblast stem cell lines. These differ from immortalized placental lines in their capacity to differentiation into several cytotrophoblast subtypes including terminal differentiation of endovascular cells. Cell cultures therefore closely mimic the implantation process iii vitro and represent an important new model of placental dysfunctions such as pre-eclampsia. The efficient generation of endovascular cytotrophoblast also offers the prospect of using these cells for regenerative medicine. Their pseudo-vasculogenic and invasive characteristics might be utilised in a variety of cell therapies remote from the uterus but related to angiogenesis and vessel remodelling, especially as expression of HLA-G (17) and indoeamine 2,3, -dioxygenase (29,30) render the cells naturally refractory to immune rejection.

### REFERENCES

1. B. Paria et al., Science 296, 2185 (2002).
2. S. Laird et al., Human Reprod. Update 9, 163 (2003).
3. K. Red-Horse et al., J Clin. Invest. 114, 744 (2004).
4. S. Tanaka et al., Science 282, 2072 (1998).
5. J. Henderson J. et al., Stem Cells 20, 329 (2002).
6. B. Gerami-Naimi et al., Endocrinology 145, 1517 (2004).
7. R. Xu et al., Nat. Biotecht. 20, 1261 (2002) 210.
8. M. Matin et al., Stem Cells 22, 659 (2004).
9. J. Thomson el al., Science 282, 1145 (1998).
10. R. Nishimura et al., Jpn. J. Cancer Res. 80, 968 (1989).
11. T. Niwa et al., Mol. Cell Biol. 22, 1526 (2002).
12. E. Kani et al., Hum. Reprod. 14, 2131 (1999).
13. T. Haiglh et al., Placenta 20, 615 (1999).
14. A. King, L. Thomas, P. Bischof Placenta 21, S113 (2000).
15. T. Nagamatsu et al., Placenta 25, 153 (2004).
16. I. Ginis et al., Dev. Biol. 269, 360 (2004).
17. Y. Loke, King, A. in Human Implantation, cell biology and immunology, (Cambridge University Press, Cambridge, 1995). pp 82-101.
18. S. Kovats et al., Science, 248, 220 (1990).
19. S. Levenberg et al., Proc. Natl. Acad. Sci. 99, 4391 (2002).
20. D. Kaufman et al., Blood 103, 1325 (2004).
21. C. Diamsky, S. Fisher, Curr. Opin. Cell Biol. 10, 660 (1998).
22. T. Korff, T. Krauss, H. Augustin, Ex. Cell Res. 297, 415 (2004).
23. S. Laird, T. Li A. Bolton, Hum. Reprod. 8, 795 (1993).
24. Carver et al., Hum. Reprod. 18, 283 (2003).
25. E. Staun-Ram et al., Reprod. Biol. Endocrin. 2, 59 (2004)
26. S. Campbell et al., Biol. Reprod. 71, 244 (2004)
27. M. Amit et al., Dev. Biol 227, 271 (2000).
28. H, Niwa, J. Miayazalci, A. Smith, Nat. Gen. 24, 372 (2000).
29. D. Munn et at, Science 297,1867 (2002)
30. A. Honig et al., J. Reprod. Immunol., 61, 79 (2004).

1. An isolated cytotrophoblast stem cell wherein said stem cell expresses HLA-G and HLA class I antigen.

2. A stem cell according to claim 1, wherein said stem cell is mononticlear.

3. A stem cell according to claim 1, wherein said stem cell expresses at least one stem cell marker selected from the group consisting of: cytokeratin 7; stage specific embryonic antigen 1; human placental lactogen; caudal related homeobox; vimentin; and Cd9.

4. A stem cell according to claim 1, wherein said stem cell is isolated from a primate.

5. A stem cell according to claim 4, wherein said primate is human.

6. A stem cell according to claim 1, wherein said stem cell is genetically modified.

7. (canceled)

8. A culture comprising a cytotrophoblast stem cell according to claim 1, wherein said culture is contained within a cell culture vessel.

9. A spheroid body comprising a cytophoblast stem cell according to claim 1 and a collagen based cell support matrix.

10. A spheroid body according to claim 9, wherein said tissue is isolated from a primate.

11. A spheroid body according to claim 10, wherein said primate is a human.

12. (canceled)

13. A spheroid body according to claim 9, wherein said cytotrophoblast stem cell in said spheroid body expresses at least one metalloprotease.

14. A spheroid body according to claim 13, wherein said metalloprotease is metalloprotease 2.

15. A method to derive human cytotrophoblast stem cells comprising selectively enriching for cytotrophoblast stem cells that express HLA-G and HLA class 1 antigen.

16. A method to derive human cytotrophoblast stem cells from embryonic stem cells comprising:
i) forming embryoid bodies comprising cylotrophoblast cells in a cell culture vessel;
ii) identifying those embryoid body cultures which produce greater than about 500 mI.U./ml chorionic gonadotrophin;
iii) culturing the embryoid bodies identified in (ii) in conditioned media from fibroblast feeder cells;
iv) pooling those embryoid bodies which produce greater than about 500 mI.U./ml chorionic gonadotrophin and disaggregating said embryoid bodies; and
v) repeating (iv) until substantially all embryoid bodies produce high levels of chorionic gonadotrophin.

17. A method according to claim 16, wherein said conditioned media comprises fibroblast growth factor 4 and heparin.

18. Spent medium produced by a method that comprises culturing cytotrophoblast stem cells selected from the group consisting of:
cytotrophoblast stem cells that express the HLA-G and HLA class 1 antigen; and cytotrophoblast stem cells that express greater than about 500 mI.U./ml chorionic gonadotrophin in culture.

19. A method to produce endovascular cytotrophoblast cells comprising:
i) providing a preparation of cytotrophoblast stem cells according claim 1;
ii) selecting from said preparation a population of cells that express both HLA-G and platelet endothelial cell adhesion molecule-1.

20. A method according to claim 19, wherein said selected cells further express Von Willebrand Factor.

21. A method according to claim 19, wherein said preparation is cultured under high oxygen tension.

22. An endovascular cytotrophoblast cell obtained or obtainable by a method comprising:
i) providing a preparation of cytotrophoblast stem cells according claim 1:
ii) selecting from said preparation a population of cells that express both HLA-G and platelet endothelial cell adhesion molecule-1.

23. An in vitro method for the formation of spheroids comprising cytotrophoblast stem cells comprising:
i) providing a cell culture vessel comprising:
a) cytotrophoblast stem cells according to claim 1;
b) cell culture medium; and
ii) providing conditions which promote the growth and differentiation of said cytotrophoblast stem cells in said spheroid.

24. Spent medium produced by culturing spheroids comprising cytotrophoblast stem cells according to an in vitro method comprising:
providing a cell culture vessel comprising:
a) cytotrophoblast stem cells according to claim 1;
b) cell culture medium; and
ii providing conditions which promote the growth and differentiation of said cytotroblast stem cells in said spheroid.

25. A method for the identification of genes associated with cytotrophoblast stem cell differentiation comprising:
providing a preparation comprising at least one cytotrophoblast stem cell according to claim 1:
i) extracting nucleic acid from said cell preparation;
ii) contacting said extracted nucleic acid with a nucleic acid array; and
iii) detecting a signal which indicates the binding of said nucleic acid to a binding partner on said nucleic acid array.

26. A method according to claim 25 wherein said method includes the additional steps of:
i) collating the signal(s) generated by the binding of said nucleic acid to said binding partner;
ii) converting the collated signal(s) into a data analysable form.

27. A method according to claim 25, wherein said method includes a comparison of the array signal produced between different populations of cytotrophoblast stem cells isolated from different animal subjects.

28. A method for the preparation of a library comprising cytotrophoblast stem cell specific gene expression products comprising:
i) providing a preparation comprising a cytotrophoblast stem cell according to claim 1 any of claims 1-6;
ii) extracting nucleic acid from said cell preparation;
iii) preparing a cDNA from ribonucleic acid contained in said extracted nucleic acid; and
iv) ligating CDNA formed in (iii) into a vector.

29. A method according to claim 28, wherein said vector is a phage based vector.

30. An in vitro method to analyze the invasive properties of cytotrophoblast stem cells comprising:
i) providing a spheroid according to claim 9 and endometrial tissue; and
ii) monitoring the invasive phenotype of cytotrophoblast cells with respect to said endometrial tissue.

31. A method to identify agents that modulate the angiogenic activity of endothelial cells comprising:
i) providing a preparation of endovascular cytotrophoblast cells according to claim 22 and an agent to be tested;
ii) determining the effect, o f the agent on the proliferation and/or motility of said endovascular cytotrophoblast cells.

32. A method according to claim 31 wherein said agent is an antagonist.

33. A method according to claim 31 wherein, said agent is an agonist.

34-37. (canceled)

38. A composition comprising an isolated mammalian cytotrophoblast stem cell, or a cell derived from a cytotrophoblast stem cell, and at least one further isolated mammalian cell that is not a mammalian cytotrophoblast stem cell.

39. A composition according to claim 38 wherein said mammalian cell is a human cell.

40. A composition according to claim 39 wherein said mammalian cell is selected from the group consisting of: an epidermal keratinocyte; a fibroblast cell; an epithelial cell; a neuronal glial cell; neural cell; a hepatocyte stellate cell; a mesenchymal cell; a muscle cell; a kidney cell; a blood cell; a pancreatic β cell; or an endothelial cell.

41. A composition according to claim 40 wherein said cell is a pancreatic β cell.

42. A composition according to claim 39 wherein said mammalian cell is a stem cell.

43. A composition according to claim 42 wherein said stem cell is selected from the group consisting of: a haemopoictic stem cell; a neural stem cell; a bone stem cell; a muscle stem cell; a mesenchymal stem cell; an epithelial stem cell; an endodermal stem cell; an embryonic stem cell; an embryonic germ cell.

44. A composition according to claim 43 wherein said mammalian cell is an embryonic stem cell or an embryonic germ cell.

45. A composition according to claim 38 wherein said mammalian cell and said cytotrophoblast stem cell are autologous.

46. A composition according to claim 38 wherein said composition comprises an additional agent wherein said agent is an immunosuppressant.

47. A vehicle wherein said vehicle includes a mammalian cytotrophoblast stem cell, or a cell derived from a cytotrophoblast stem cell according to claim 1, and at least one further isolated mammalian cell that is not a mammalian cytotrophoblast stem cell.

48. A method to modulate cell/tissue rejection in transplantation therapy comprising:
surgically inserting into an animal a cytotrophoblast stem cell, or a cell derived from a cytotrophoblast stem cell according to claim 1 and at least one further mammalian cell;

49. A method according to claim 48 wherein said mammalian cell is a human cell.

50. A method according to claim 48 wherein said cell is selected from the group consisting of: an epidermal keratinocyte; a fibroblast; an epithelial cell; a neuronal glial cell or neural cell; a hepatocyte stellate cell; a mesenchymal cell; a muscle cell; a kidney cell; a blood cell; a pancreatic β cell; or an endothelial cell.

51. A method according to claim 50 wherein said cell is a pancreatic β cell.

52. A method according to claim 50 wherein said mammalian cell is a stem cell.

53. A method according to claim 52 wherein said stem cell is selected from the group consisting of: a haemopoietic stem cell; a neural stem cell; a bone stem cell; a muscle stem cell; a mesenchymal stem cell; an epithelial stem cell; an endodermal stem cell; a pluripotent embryonic stem cell; an pluripotent embryonic germ cell.

54. A method according to claim 53 wherein said mammalian cell is a pluripotent embryonic stem cell or a pluripotent embryonic germ cell.

55. A method according to claim 48 wherein said mammalian cell and said cytotrophoblast cell are autologous.

56. An isolated chimeric cell wherein said cell is the product of a fusion between a first cell, or part thereof, which is a cytotrophoblast stem cell according to claim 1 and a second cell wherein said first and second cell are derived from the same species.

57. A chimeric cell according to claim 56 wherein said cell comprises a cytoplasmic part derived from a cytotrophoblast stem cell and a nucleus derived from a cell that is not a cytotrophoblast stem cell.

58. A chimeric cell according to claim 56 wherein said cell comprises a nucleus derived from a cytotrophoblast stem cell and a cytoplasmic part derived from a cell that is not a cytotrophoblast cell.

59. A chimeric cell according to claim 56 wherein the first and second cells are human cells.

60. A chimeric cell according to claim 56 wherein said second cell is selected from the group consisting of: an epidermal keratinocyte; a fibroblast; an epithelial cell; a neuronal glial cell or neural cell; a hepatocyte stellate cell; a mesenchymal cell; a muscle cell; a kidney cell; a blood cell; a pancreatic β cell; or an endothelial cell.

61. A cell culture comprising a chimeric cell according to claim 56.

62. A method for the production of a chimeric cell comprising the steps of:
forming a preparation comprising a first cell which is a cytotrophoblast stem cell according to claim 1 and a second cell wherein said first and second cells are derived from the same species; and
providing conditions wherein said first and second cells fuse to form a chimeric cell.

63. A chimeric cell according to claim 56 for use in the manufacture of a cell composition for the modulation of cell/tissue rejection in transplantation therapy.

64. A method to treat a condition that would benefit from transplantation therapy comprising administering a chimeric cell according to claim 56.

65. (canceled)

66. The method of claim 48, further comprising monitoring the immune status of the animal as a measure of the acceptance or otherwise of said mammalian cell.

67. The method according to claim 21, wherein the high oxygen tension culturing comprises at least 5% CO₂.

68. The method of claim 26, further comprising;
i) providing an output for the analyzed date.

69. An isolated proliferating primate cytotrophoblast stem cell wherein said stern cell is mononuclear and expresses HLA-G and cytokeratin 7.
